(19) United States
(12) Patent Application Publication
(10) Pub. No.: US 2019/0125660 A1
(43) Pub. Date: May 2, 2019

(54) Orally dissolving mucoadhesive films utilizing menthol and l-arginine to enhance the bioavailability of cannabinoids

(71) Applicants: Ronald J. Thompson, Cincinnati, OH (US); James M. Thompson, Cincinnati, OH (US)

(72) Inventors: Ronald J. Thompson, Cincinnati, OH (US); James M. Thompson, Cincinnati, OH (US)

(73) Assignee: Calitas Therapeutics, Inc, Newport, KY (US)

(21) Appl. No.: 16/350,011

(22) Filed: Sep. 12, 2018

Related U.S. Application Data
(60) Provisional application No. 62/579,212, filed on Oct. 31, 2017.

(51) Int. Cl. A61K 9/00; A61K 47/10; A61K 47/18; A61K 31/05; A61K 31/352

(52) U.S. Cl. CPC A61K 9/006; A61K 47/10; A61K 31/352; A61K 31/05; A61K 47/183

(57) ABSTRACT

A method for providing an orally dissolvable composition of menthol, 1-arginine and one or more cannabinoids for quickly and reliably providing enhanced pharmacokinetic bioavailability of the cannabinoids to a mammalian subject. The orally dissolvable composition is preferably in the form of a thin orally dissolving mucoadhesive strip or film that is placed in the buccal cavity or under the tongue of a user. The menthol and 1-arginine function as permeation enhancers and local vasodilators, providing improved oral transmucosal absorption of the cannabinoids.

### FIELD OF THE INVENTION

The present invention relates in general to compositions and methods for delivering cannabinoids to mammalian subjects, and more particularly to an orally dissolving composition in the dosage form of a thin film or mucoadhesive strip comprising menthol, 1-arginine, and one or more cannabinoids, such as THC and/or CBD, for providing enhanced pharmacokinetic bioavailability of the cannabinoids, and is based upon our Provisional Application No. 62/579,212, filed 31 Oct. 2017, which application is incorporated herein by reference.

### PRIOR ART: MENTHOL AND L-ARGININE

Menthol is a mucous membrane permeation enhancer that drives penetration of 1-arginine into the submucosal tissues. L-arginine is the only substrate for the Nitric Oxide Synthase pathway that converts 1-arginine into Nitric Oxide, a potent vasodilator. There are three isoenzymes of the Nitric Oxide Synthase, eNOS (endothelial—cells that line the blood vessels), nNOS (neuronal) and iNOS (inducible). All three isoenzymes cause vasodilitation of sub mucous membrane blood vessels. In addition, the Nitric Oxide stimulates the production of cyclic GMP (guanosine mono phosphate) a very potent and long acting vasodilator.

All of the menthol and 1-arginine Prior Art references are describing female products that are genitally placed (clitoris and vulvae/vagina), manually applied, FDA-cleared (510K 021125) for increased sexual enhancement, and are not intended for oral use (FDA labeling).

U.S. Pat. No. 6,322,493 Thompson

U.S. Pat. No. 6,548,545 Thompson

U.S. Pat. No. 6,702,733 Thompson

U.S. Pat. No. 6,989,163 Thompson et al

20170239175 Thompson et al

20070060653 Thompson

20070042060 Thompson

20050245494 Thompson et al

20050186294 Thompson et al

20050100618 Thompson et al

20050069597 Thompson et al

20040258774 Thompson et al

Prior Art: Orally Dissolving Thin Films Delivering Cannaboids

U.S. Pat. No. 9,833,461 Modi, Pankaj

20160051510 Allen et al

20170290870 Schaneville, Scott

### BACKGROUND OF THE INVENTION

Cannabis or marijuana is the most widely used illicit drug in the world and has been cultivated by mankind for over 6000 years. Cannabinoids, most notably THC (tetrahydrocannabinol, the psychoactive compound in marijuana) and CBD (cannabidiol), are chemicals that are produced by Cannabis indica and Cannabis sativa plant strains. There are over 100 different cannabinoids isolated from Cannabis exhibiting varied effects. To date, two cannabinoid-specific receptors have been cloned and characterized from mammalian tissues: CB₁, particularly abundant in the brain, and CB₂, mainly expressed in the immune system. Cannabinoids mimic the action of the endogenous neurotransmitter anandamide, a naturally produced endocannabinoid. Examples of cannabinoids include, but are not limited to, Δ-9-tetrahydrocannabinol (Δ-9-THC); Δ-8-tetrahydrocannabinol (Δ-8-THC); 11-hydroxy-Δ-9-THC; Δ-9-cannabidiol (Δ-9-CBD); Δ-8-cannabidiol (Δ-8-CBD); cannabinol (CBN); levonantradol, cannabivarin (CBDV), Δ-9-tetrahydrocannabivarin (Δ-9-THCV), cannabigerol (CBG), and acids and analogs thereof.

It is now possible to synthesize many cannabinoids in the laboratory, eliminating the need to grow Cannabis plants for extraction of the compounds. Whole or crude marijuana (including marijuana oil or hemp oil) containing Δ-9-THC is regulated by the United States Drug Enforcement Administration (DEA) as a Schedule I Drug because it is a hallucinogen, and it is not approved by the US Food and Drug Administration (FDA) for any medical use. However, the use of marijuana to treat some medical conditions is legal under state laws in many states. For example, capsules of THC (dronabinol) and its synthetic analogue nabilone are approved in several countries and by the FDA to treat chemotherapy-induced nausea and vomiting, and they are commonly used to treat chemotherapy-related anxiety, loss of appetite, and neuropathic pain.

More specifically, cannabinoids such as THC have been shown to significantly stimulate appetite in patients that have cachexia related to cancer, and to significantly reduce chronic neuropathic pain where traditional treatment has been unsuccessful, without adversely affecting the efficacy of the chemotherapy. In addition, chemotherapy patients treated with THC report that food tastes better and they experience a higher quality of sleep and relaxation. One of the major cannabinoids, cannabidiol (CBD), is effective at treating the more difficult to control symptoms of nausea, as well as preventing anticipatory nausea in chemotherapy patients. Studies also report use of cannabinoids in treating the weight loss syndrome of AIDS, in reducing intraocular pressure for the treatment of glaucoma, as well as providing muscle relaxing effects in multiple sclerosis patients and anti-convulsant effects in seizure patients. Research also suggests that both THC and CBD have anti-inflammatory properties, which can reduce swelling in the hands and feet that may occur in patients undergoing chemotherapy.

There are four main methods for administering cannabinoids: topical, inhalational, oral consumption, and sublingual/buccal absorption. Each of these methods has unique benefits and potential drawbacks. Topicals include creams and oils that are infused with cannabinoids and applied to the skin. Unlike other delivery methods, topicals do not typically cause a cerebral high, and are best used for localized relief of inflammation, joint pain or sore muscles. Inhalation, typically via smoking marijuana, is historically the main method of recreational cannabinoid administration. However, there are concerns that smoking marijuana may itself be a cause of lung cancer. Indeed, marijuana smoke notoriously carries more tars and other potentially carcinogenic particulate matter than tobacco. Furthermore, many cancer patients find the act of smoking unappealing, as well as generally unhealthy. Use of vaporizers for inhalation administration of cannabinoids does not avoid the production of thermal byproducts, and also can produce tracheal and lung irritation. For these reasons, smoking is not an ideal medical means of administration of cannabinoids. Smoke-free attempts have been made to overcome some of the problems associated with smoking cannabinoids, such as smokeless inhalable aerosol formulations, but such formulations typically have varying effectiveness and have also been found to cause irritation of the trachea and lungs.

Oral consumption of Cannabis or cannabinoids has limited effectiveness in that cannabinoids, like many pharmaceutically active agents, are metabolized in the liver. As a result, oral consumption can lead to alteration, delayed onset and/or inactivation of the active ingredient before reaching its target destination, what is known as “the first pass effect.” Accordingly, sublingual and/or buccal oral dosage forms may be preferred for delivering certain pharmaceutically active agents, including cannabinoids, to the bloodstream. Buccal and sublingual oral dosage forms are designed to release the pharmaceutically active ingredient into the mouth for absorption through the oral mucosa. Mucoadhesive dosage forms such as buccal or sublingual thin strips are inserted into the buccal pouch (a space generally defined between a cheek and the gums) or held under the tongue, effecting drug release into and through the oral mucosa and minimizing release of the active ingredients into the gastrointestinal tract, thereby bypassing gastrointestinal and hepatic “first pass” metabolism processes.

While attempts have been made to administer cannabinoids by way of oral transmucosal administration (see, e.g., U.S. Pat. Nos. 9,044,390 and 9,186,386 to Speier, which are incorporated herein by reference in their entirety), to date such attempts have proven unsatisfactory in adequately and reliably delivering cannabinoids to the bloodstream. A known problem with oral transmucosal (e.g. sublingual and buccal) administration is that pharmaceutically active agents, especially those that are not rapidly absorbed through the oral mucosa, may be washed away in substantial proportion because of the continuous secretion of saliva in the oral cavity. However, while the active ingredient must be absorbed quickly and not washed away into the stomach, it must also not be absorbed so slowly as to cause discomfort or inconvenience for the patient, which can lead to non-compliance. Similarly, the dosage form should be of a size and shape that avoids discomfort to the patient, and does not leave a gritty or other undesirable feeling in the mouth.

Accordingly, the present invention is motivated by the need in the art for improved compositions and methods for administration of cannabinoids. In particular, it would be beneficial to provide a composition containing one or more cannabinoids for oral transmucosal administration in which the cannabinoids are adequately and reliably absorbed into the bloodstream. It would also be beneficial to provide a composition containing one or more cannabinoids in combination with permeation enhancers and local vasodilators in order to improve absorption of the cannabinoids. It would further be advantageous to provide an improved composition containing cannabinoids which can be delivered via an orally dissolving mucoadhesive strip or film to quickly, reliably and comfortably provide enhanced pharmacokinetic bioavailability and delivery of the cannabinoids to the patient.

### BRIEF SUMMARY OF THE INVENTION

The present invention pertains to a method of delivery of compositions to a user, the method including arranging cannabinoids in combination with permeation enhancers and local vasodilators such as menthol and 1-arginine in which the composition is in the form of an orally dissolving film or strip. The inventive composition can provide a useful means to safely deliver cannabinoids for treating the debilitating side effects of cancer chemotherapy, as well as muscle spasticity and pain associated with multiple sclerosis, weight loss associated with AIDS, increased intraocular pressure associated with glaucoma, and other symptoms.

One aspect of the invention provides a composition and the method of its assembly suitable for oral transmucosal delivery comprising at least one cannabinoid in combination with menthol and 1-arginine, wherein the composition is in the dosage form of an orally dissolving mucoadhesive film.

Another aspect of the invention provides an orally dissolving composition and a method of delivery of its assembly, the assembly comprising a therapeutically effective amount of cannabinoids Δ-9-CBD and Δ-9-THC in combination with menthol and 1-arginine, wherein the composition is in the dosage form of an orally dissolving mucoadhesive film suitable for oral transmucosal delivery of the cannabinoids.

The invention thus comprises a method for facilitating oral transmucosal delivery of a cannabinoid to an individual, the method comprising combining at least one cannabinoid with menthol and 1-arginine to form a composition, wherein the composition is in the form of an orally dissolvable mucoadhesive film; and delivering the orally dissolvable mucoadhesive film to an individual. The composition of at least one cannabinoid comprises at least one of Δ-9-tetrahydrocannabinol (Δ-9-THC); Δ-8-tetrahydrocannabinol (Δ-8-THC); 11-hydroxy-Δ-9-THC; Δ-9-cannabidiol (Δ-9-CBD); Δ-8-cannabidiol (Δ-8-CBD); cannabinol (CBN); levonantradol, cannabivarin (CBDV), Δ-9-tetrahydrocannabivarin (Δ-9-THCV), cannabigerol (CBG), and acids and analogs thereof. The composition of at least one cannabinoid which comprises a therapeutically effective amount of Δ-9-cannabidiol (Δ-9-CBD). The composition of the at least one cannabinoid comprises a therapeutically effective amount of Δ-9-tetrahydrocannabinol (Δ-9-THC). The composition of the at least one cannabinoid comprises a therapeutically effective amount of both Δ-9-CBD and Δ-9-THC. The orally dissolving mucoadhesive strip comprises a polymeric carrier matrix.

The invention also includes a method of producing an orally dissolving composition comprising the steps of: combining a therapeutically effective amount of cannabinoids Δ-9-CBD and Δ-9-THC with menthol; and 1-arginine, wherein the composition is in the dosage form of an orally dissolving mucoadhesive film suitable for oral transmucosal delivery of the cannabinoids.

The nature and advantages of the present invention will be more fully appreciated after reviewing the accompanying drawings, detailed description and claims.

### DETAILED DESCRIPTION OF THE INVENTION

As used herein, the term “cannabinoid” refers to a class of diverse chemical compounds that act on mammalian cannabinoid receptors such as CB₁ and CB₂, and include endocannabinoids (such as anandamide, produced naturally in the body), phytocannabinoids (found in Cannabis plants and some other plants), and synthetic cannabinoids (manufactured chemically). The most notable cannabinoid is THC (Δ-9-tetrahydrocannabinol), the primary psychoactive compound of Cannabis. Cannabidiol (CBD) is another major constituent of the Cannabis plant, representing up to 40% in extracts of the plant resin.

As used herein, the terms “mucoadhesion” and “mucoadhesive” refer to an adhesive property or effect in which a product binds to the mucin layer of a biological membrane, such as the oral mucosa of the mouth. Mucoadhesion is associated with benefits such as controlled, sustained release, prolonged residence time at the site of action, the ability to target specific mucosa, and ease of application which leads to higher rates of patient compliance. For example, a mucoadhesive strip according to the present invention can be a sheet or film which adheres to the mucosal surface of the mouth and is difficult to remove once placed in the mouth, which helps achieve optimum absorption of the pharmaceutically active ingredient.

As used herein, the terms “strip” and “film” refer to sheets comprising a polymeric carrier matrix, in any shape, including rectangular, square, or other desired shape. The films described herein are typically thin films, but may be any desired thickness and size so long as they can be placed into the oral cavity of the user. Films may be in a single layer or they may be multi-layered, including laminated films.

The present invention provides a method for providing an orally deliverable composition, the composition comprising at least one cannabinoid in combination with menthol and 1-arginine, wherein the composition is in the form of an orally dissolving mucoadhesive strip. The inventive composition is specifically indicated for patients in need of cannabinoid therapy, such as therapy employing at least one of Δ-9-CBD, Δ-9-THC, Δ-8-THC, Δ-8-CBD, 11-hydroxy-Δ-9-THC, cannabinol (CBN), levonantradol, cannabivarin (CBDV), Δ-9-tetrahydrocannabivarin, cannabigerol (CBG), and acids and analogs thereof. In a preferred embodiment, the composition comprises a therapeutically effective amount of Δ-9-cannabidiol (Δ-9-CBD). In another preferred embodiment, the composition comprises a therapeutically effective amount of Δ-9-tetrahydrocannabinol (Δ-9-THC).

Sublingual and buccal delivery allows the mucoadhesive strip or film containing the components of the inventive composition to adhere to the oral mucosa and dissolve in the immediate vicinity where the product is placed. This allows the menthol and 1-arginine to enhance the vascular permeability of the oral mucosa and allow for enhanced absorption of the cannabinoid into the bloodstream to rapidly exert its pharmacological effect. Such sublingual/buccal delivery of the inventive composition is more effective than oral dosing because it bypasses the acidic environment, gastric juices and enzymes in the stomach and gastrointestinal tract, as well as bypassing first pass metabolism in the liver. The highly vascular mucosal lining between the cheek and gum, or under the tongue where the mucoadhesive strips are placed is an ideal and convenient location for the cannabinoid to be absorbed.

The present invention allows a method for delivery of various dosages of cannabinoids to a user, such as between about 0.5 and 10 mg of cannabinoid per unit dosage form. Patients typically can be administered the cannabinoid in dosages of 1 mg to 10 mg per dose, and between 2 and 6 times daily, until the symptoms being treated subside (e.g. nausea/vomiting, appetite, chronic neurogenic pain, muscle spasm, glaucoma, etc.). The maximum dosage of cannabinoid administered to a patient is typically 20 mg/dose. The orally dissolving, mucoadhesive strips can have a microporous, porous, or honeycomb design which can absorb a preferred cannabinoid extract such as THC or CBD, for medicinal use. Moreover, the smaller total dose of cannabinoid ranging from 0.5 to 10 mg/dose for therapeutic effect also lends itself for effective dosage design dictated by the small physical size of each mucoadhesive strip.

Menthol is a lipophilic mucus membrane permeation enhancer that, for purposes of the present invention, improves the diffusion of 1-arginine and cannabinoids across the oral mucus membrane barrier, and aids in the absorption of cannabinoids across the oral mucosa. Menthol is only functional as a permeation enhancer for a short duration, i.e. about 1 minute to 5 minutes. This is well explained, for example, in U.S. Pat. No. 6,702,733 to Thompson, which is incorporated herein by reference in its entirety. The use of 1-arginine in the inventive composition induces the nitric oxide synthase enzyme to produce nitric oxide (NO) and cyclic GMP, which induces prolonged vasodilation. The rate limiting factor of the induction of the nitric oxide synthase enzyme is the availability of 1-arginine. By extending the 1-5 minutes of vasodilation provided by menthol to about 20-40 minutes, 1-arginine can allow an extended period of time for the cannabinoid(s) to be absorbed into the oral mucosal vasculature.

More specifically, the menthol component of the inventive deliverable assembly composition functions as a mucus membrane permeation enhancer which causes transient vasodilation and allows the 1-arginine and cannabinoids to easily and rapidly enter the oral mucus membranes. The absorbed 1-arginine then induces production of nitrous oxide (NO) in oral mucosal cells, which diffuses to neighbor cells and reaches its target, guanylate cyclase. The activation of guanylate cyclase induces an increase in cyclic guanylate monophosphate (cGMP), which is a signaling messenger that relaxes smooth muscle tissues and leads to vasodilation and increased blood flow. The immediate, increased and prolonged blood flow provided by the combination of menthol and 1-arginine provides enhanced pharmacokinetic bioavailability of the cannabinoids.

Dosage forms envisioned for the present invention, as initially noted above, include orally dissolving mucoadhesive films or strips made of a polymeric carrier matrix impregnated with or including cannabinoid(s)+menthol+1-arginine. The inventive strips are intended to be flexible, quickly wettable, and non-irritating to the user, and they are also intended to dissolve rather quickly while providing an adequate level of mucoadhesion. For the present invention, it is preferable to use films that provide a quick enough dissolution rate, most desirably between about 1 minute and about 20 minutes, while providing an acceptable mucoadhesion level such that the film is not easily removable once it is placed in the oral cavity of the user.

Mucoadhesive strips are generally known in the art, for example, as disclosed in U.S. Pat. Nos. 8,475,832, 8,663,687 and 9,511,033 to Myers et al., which are incorporated herein by reference in their entirety. A preferred mucoadhesive polymeric carrier matrix for use in strips or films containing the inventive composition can include a synthetic polymer such as, but not limited to, polyacrylic acid, polyethylene oxide (Polyox), polymethacrylate derivatives, polycarbophil, poloxamer mixtures, Carbopol, hydroxy-methylcellulose, hydroxy-propylcellulose, hydroxypropylmethyl-cellulose (HPMC), and polyethylene glycol (PEG), as well as naturally occurring polymers such as hyaluronic acid and chitosan, alone or in combination.

While the present invention has been illustrated by the description of embodiments thereof in considerable detail, it is not intended to restrict or limit the scope of the appended claims to such detail. Additional advantages and modifications will be readily apparent to those skilled in the art. Departures may be made from such details without departing from the scope of the invention.

What is claimed is:

1. A method for facilitating oral transmucosal delivery of a cannabinoid to an individual, the method comprising:
combining at least one cannabinoid with menthol and 1-arginine to form a composition, wherein the composition is in the form of an orally dissolvable mucoadhesive film; and
delivering the orally dissolvable mucoadhesive film to an individual.

2. The method of claim 1, wherein the composition of the at least one cannabinoid comprises at least one of Δ-9-tetrahydrocannabinol (Δ-9-THC); Δ-8-tetrahydrocannabinol (Δ-8-THC); 11-hydroxy-Δ-9-THC; Δ-9-cannabidiol (Δ-9-CBD); Δ-8-cannabidiol (Δ-8-CBD); cannabinol (CBN); levonantradol, cannabivarin (CBDV), Δ-9-tetrahydrocannabivarin (Δ-9-THCV), cannabigerol (CBG), and acids and analogs thereof.

3. The method of claim 1, wherein the composition of the at least one cannabinoid comprises a therapeutically effective amount of Δ-9-cannabidiol (Δ-9-CBD).

4. The method of claim 1, wherein the composition of the at least one cannabinoid comprises a therapeutically effective amount of Δ-9-tetrahydrocannabinol (Δ-9-THC).

5. The method of claim 1, wherein the composition of the at least one cannabinoid comprises a therapeutically effective amount of both Δ-9-CBD and Δ-9-THC.

6. The method of claim 1, wherein the orally dissolving mucoadhesive strip comprises a polymeric carrier matrix.

7. A method of producing an orally dissolving composition comprising the steps of:
combining a therapeutically effective amount of cannabinoids Δ-9-CBD and Δ-9-THC with menthol and 1-arginine, wherein the composition is in the dosage form of an orally dissolving mucoadhesive film suitable for oral transmucosal delivery of the cannabinoids.
